(19) United States
(12) Patent Application Publication
(10) Pub. No.: US 2022/0287756 A1
(43) Pub. Date: Sep. 15, 2022

(54) APPARATUS FOR REMOVING SURGICAL POLYGONAL LOCKING SCREW

(71) Applicants: CHUNGNAM NATIONAL UNIVERSITY HOSPITAL, Daejeon (KR); THE INDUSTRY & ACADEMIC COOPERATION IN CHUNGNAM NATIONAL UNIVERSITY (IAC), Daejeon (KR)

(72) Inventors: Jeong-kil LEE, Daejeon (KR); Chan KANG, Daejeon (KR)

(73) Assignees: CHUNGNAM NATIONAL UNIVERSITY HOSPITAL, Daejeon (KR); THE INDUSTRY & ACADEMIC COOPERATION IN CHUNGNAM NATIONAL UNIVERSITY (IAC), Daejeon (KR)

(21) Appl. No.: 17/625,583

(22) PCT Filed: Jul. 9, 2020
(86) PCT No.: PCT/KR2020/009026

§ 371 Date: Jan. 7, 2022

(30) Foreign Application Priority Data
Jul. 9, 2019 (KR) 10-2019-0082819

(51) Int. Cl. A61B 17/92; A61B 17/88; A61B 17/90

(52) U.S. Cl. CPC A61B 17/92; A61B 17/888; A61B 17/90

(57) ABSTRACT

A surgical polygonal locking screw removal device includes: a body including a passage part in which a passage having a preset diameter is formed, and a polygonal expansion part configured to be coupled to a polygonal groove formed in a surgical locking screw to remove the surgical locking screw, the polygonal expansion part protruding from a leading end of the passage part; and a handle configured to be inserted into a rear end of the passage part and screw-coupled to the passage part, and configured to expand the polygonal expansion part.

### TECHNICAL FIELD

The present disclosure relates to a surgical polygonal locking screw removal device.

### BACKGROUND

In the orthopedic field, a commonly used instrument for performing fracture and bone correction surgery is a locking compression plate and a polygonal locking screw used therefor. In this case, a hexagonal screw is generally used as the polygonal locking screw.

After sufficient bone union has occurred through these surgeries or procedures, it may be necessary to remove the polygonal locking screws and a metal plate. Even if bone union does not occur, when circumstances such as infection or metal plate breakage occur, the metal material must be removed.

Recently, a metal plate of the most used type in the orthopedic field is a locking compression metal plate, which has many advantages compared to other types of metal plates, and thus its use is increasing.

However, the biggest problem in using the metal plate and the polygonal locking screw is that a head of the polygonal locking screw is damaged when performing the removal of the metal material.

When the metal locking screw is fixed to the bone and bone union proceeds after a certain period of time, the bonding force between the bone and the screw increases. In this case, when the polygonal locking screw is removed, the high coupling force between the screw and the bone may not be overcome, and thus the polygonal groove formed in the screw may be crushed.

If the polygonal groove formed in the head of the locking screw is damaged, it is difficult to remove the screw with a general screwdriver, so that the screw needs to be removed by using a conventional polygonal locking screw removal driver or if this method fails, the screw should be removed after grinding the head of the screw with a special equipment.

In using the conventional polygonal locking screw removal driver, the polygonal locking screw is removed by coupling the driver to a polygonal groove formed in the head of the polygonal locking screw. In this case, the polygonal locking screw can be loosened by rotating the screw removal driver with a constant force in a direction while matching the screw removal driver exactly perpendicularly in the polygonal groove of the head of the screw.

However, in the conventional polygonal locking screw removal driver, when removing the polygonal locking screw, there is inevitably a gap between the screw removal driver and the polygonal groove of the head of the screw. Due to this gap, the contact force between the screw and the screw removal driver is lowered, which makes it difficult to remove the screw, and the screw removal driver may be damaged after several uses.

In addition, in the conventional polygonal locking screw removal driver, the polygonal groove formed in the head of the screw may be damaged as the contact force is lowered due to the gap between the screw and the screw removal driver, so that small metal pieces may remain in a patient's body in the process of removing the screw head and the metal plate with a special tool. The small metal pieces may cause inflammation or infection as foreign substances.

### SUMMARY

An embodiment of the present disclosure is to provide a surgical polygonal locking screw removal device capable of easily removing a polygonal locking screw by increasing a contact force with the polygonal locking screw when removing the polygonal locking screw.

In accordance with an embodiment of the present disclosure, there is provided a surgical polygonal locking screw removal device including: a body including a passage part in which a passage having a preset diameter is formed, and a polygonal expansion part configured to be coupled to a polygonal groove formed in a surgical locking screw to remove the surgical locking screw, the polygonal expansion part protruding from a leading end of the passage part; and a handle configured to be inserted into a rear end of the passage part and screw-coupled to the passage part, and configured to expand the polygonal expansion part.

The polygonal expansion part may be formed to be divided at regular intervals along a circumferential direction so that the polygonal expansion part is radially expandable when inserted into the polygonal groove of the surgical locking screw.

Cutouts may be formed in the leading end of the passage part.

The polygonal expansion part may include a tapered portion which communicates with the passage part and an inner diameter of which is gradually decreased toward a leading end of the polygonal expansion part.

The handle may include: a rotating part configured to be gripped by a hand; a stem part connected to the rotating part, and configured to be inserted into the rear end of the passage part and screw-coupled to the passage part to move forward or backward along the passage part when the rotating part is rotated; and a pressing part provided at a leading end of the stem part and configured such that when the pressing part reaches the tapered portion of the polygonal expansion part as the stem part moves forward, the pressing part radially presses the tapered portion to expand the polygonal expansion part to be close contact with the polygonal groove.

The stem part may be screw-coupled to the passage part in a left-handed manner to move forward when the rotating part is rotated counterclockwise.

Each of the cutouts and gaps formed between the divided portions of the polygonal expansion part may be arranged on a line.

According to the embodiment of the present disclosure, when removing the surgical polygonal locking screw in the orthopedic area, the surgical polygonal locking screw removal device can easily remove the polygonal locking screw without damaging the polygonal locking screw by increasing the contact force with the polygonal locking screw.

### BRIEF DESCRIPTION OF THE DRAWINGS

FIG. 1 is a diagram illustrating a surgical polygonal locking screw removal device according to one embodiment of the present disclosure.

FIG. 2 is a view showing a state in which the surgical polygonal locking screw removal device according to one embodiment of the present disclosure is coupled to a surgical polygonal locking screw.

FIG. 3 is a reference view showing a state before a polygonal expansion part inserted into a polygonal groove of the locking screw is expanded.

FIG. 4 is a reference view showing a state in which the polygonal expansion part is expanded in the polygonal groove of the locking screw.

FIG. 5 is a view showing a state before the polygonal expansion part of the surgical polygonal locking screw removal device according to one embodiment of the present disclosure is expanded.

FIG. 6 is a view showing a state after the polygonal expansion part of the surgical polygonal locking screw removal device according to one embodiment of the present disclosure is expanded.

FIG. 7 is a cross-sectional view taken along line VII-VII of FIG. 3.

FIG. 8 is a cross-sectional view taken along line VIII-VIII of FIG. 4.

### DETAILED DESCRIPTION

Hereinafter, configurations and operations according to embodiments of the present disclosure will be described in detail with reference to the accompanying drawings. The description below is one of many aspects of the present disclosure that are patent-claimable, and the following description may form a part of the detailed description of the present disclosure.

However, detailed descriptions of known configurations or functions in describing the present disclosure may be omitted to clarify the present disclosure.

Since the present disclosure may be changed in various ways and include various embodiments, specific embodiments will be illustrated in the drawings and described in the detailed description. However, this is not intended to limit the present disclosure to a specific embodiment, and should be understood to include all changes, equivalents, or substitutes included in the spirit and technical scope of the present disclosure.

Terms including ordinal numbers, such as first and second, may be used for describing various elements, but the corresponding elements are not limited by these terms. These terms are only used for the purpose of distinguishing one element from another element.

When an element is referred to as being ‘connected’ to, or ‘accessed’ to another element, it should be understood that the element may be directly connected to, or accessed to another element, but that other elements may exist in the middle.

The terms used in the present application are only used to describe specific embodiments, and are not intended to limit the present disclosure. Singular expressions include plural expressions unless they are clearly defined differently in context.

Hereinafter, one embodiment of the present disclosure will be described in detail with reference to the accompanying drawings.

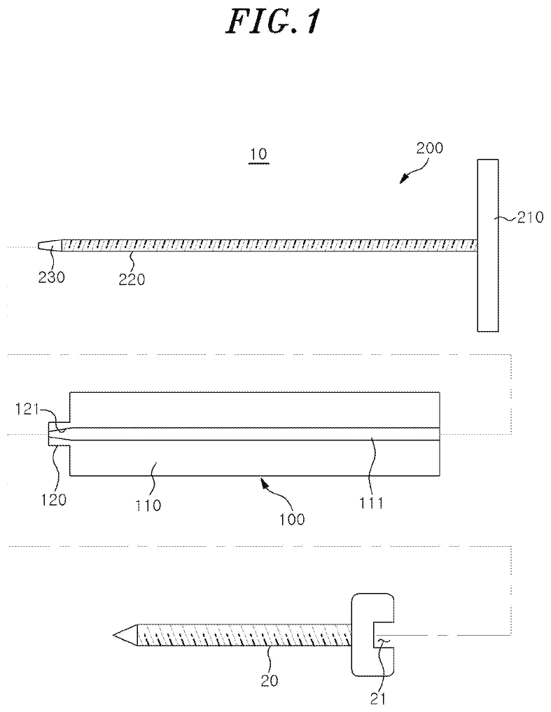

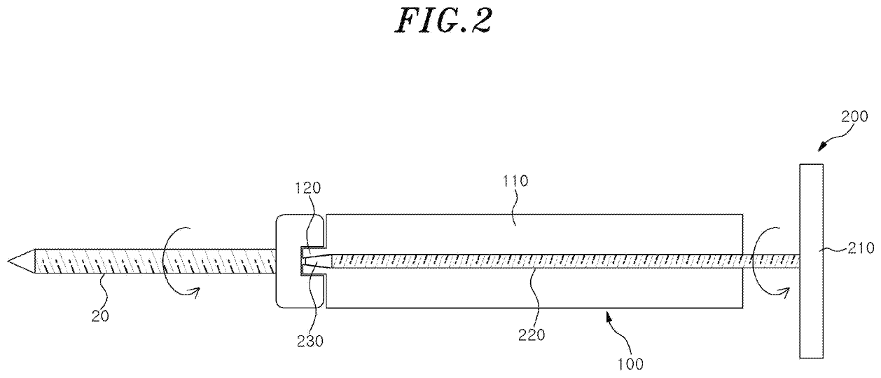

Referring to FIGS. 1 and 2, a surgical polygonal locking screw removal device 10 according to one embodiment of the present disclosure may generally include a body 100 and a handle 200.

The body 100 may be coupled in a polygonal groove 21 formed in a head of a metallic polygonal locking screw 20 (hereinafter, referred to as “locking screw 20”) fixed to a bone in the orthopedic field in order to remove the locking screw 20 from the bone by rotating locking screw 20 in a releasing direction.

The body 100 may be formed in a substantially cylindrical shape, and may be formed in a polygonal shape, such as a hexagon or an octagon, if necessary.

Specifically, the body 100 may include a passage part 110 and a polygonal expansion part 120.

The passage part 110, as shown in FIG. 1, may include therein a passage 111 extending along a longitudinal direction of the body 100 and having a predetermined diameter. The diameter of the passage 111 of the passage part 110 may be set to correspond to an outer diameter of the handle 200 inserted into the passage part 110. The passage part 110 may have a screw thread formed on its inner surface.

The polygonal expansion part 120 is inserted into the polygonal groove 21 of the locking screw 20 to rotate the locking screw 20 in a direction in which the locking screw 20 is loosened. The polygonal expansion part 120 is formed to protrude to a predetermined length from a leading end of the passage part 110, and is in close contact with the polygonal groove 21 of the locking screw 20 after being inserted into the polygonal groove 21 of the locking screw 20 to apply a rotational force to the locking screw 20. Here, the leading end may be one end in the longitudinal direction of the body 100.

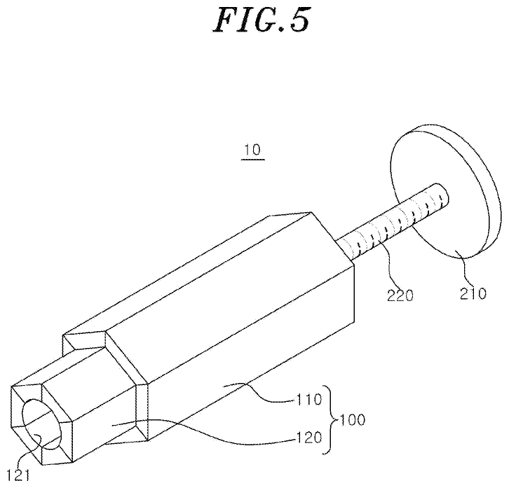

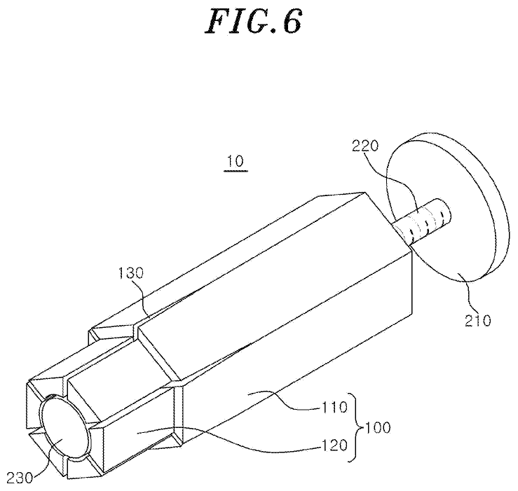

Accordingly, the polygonal expansion part 120 may be formed in a polygonal shape to correspond to the polygonal groove 21 as shown in FIGS. 5 and 6. In addition, although the polygonal groove 21 and the polygonal expansion part 120 are hexagonal in the present embodiment, the polygonal groove 21 and the polygonal expansion part 120 may have another polygonal shape and may be octagonal, for example.

The polygonal expansion part 120 may be configured to be expandable radially so as to be in close contact with the polygonal groove 21 after the polygonal expansion part 120 is inserted into the polygonal groove 21 of the locking screw 20. Accordingly, the polygonal expansion part 120 may be formed to be divided at regular intervals along a circumferential direction. Gaps may be formed between the divided portions of the polygonal expansion part 120.

In addition, cutouts 130 may be formed at the leading end of the passage part 110 so that the passage part 110 is divided along the polygonal expansion part 120 when the polygonal expansion part 120 is expanded radially. The cutouts 130 and the gaps formed between the divided portions of the polygonal expansion part 120 may be arranged linearly.

Accordingly, as shown in FIGS. 5 and 6, when the polygonal expansion part 120 is expanded radially to increase the outer diameter of the polygonal expansion part 120, the leading end of the passage part 110 is also divided by the cutouts 130, which allows the polygonal expansion part 120 to naturally expand.

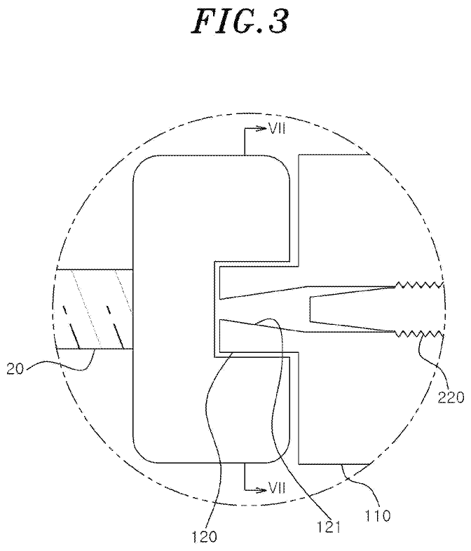

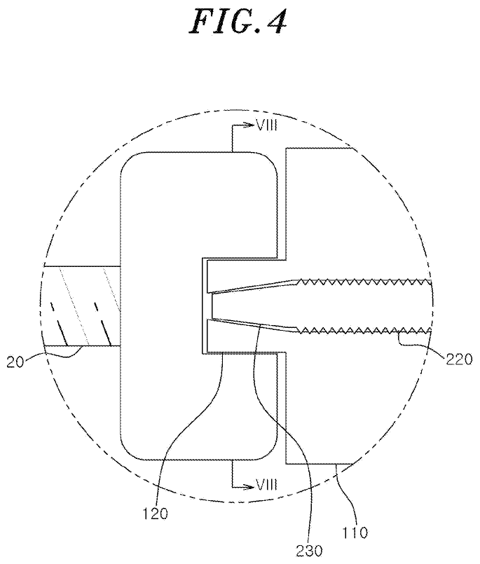

In addition, as shown in FIGS. 3 and 4, the polygonal expansion part 120 may include therein a tapered portion 121 which communicates with the passage 111 of the passage part 110 and an inner diameter of which is gradually decreased toward a leading end of the polygonal expansion part 120.

The tapered portion 121 is configured such that a diameter thereof is gradually reduced, and the polygonal expansion part 120 may be radially expanded by the tapered portion 121. The tapered portion 121 will be described again later.

In addition, referring to FIGS. 1 and 2, the handle 200 is inserted into the body 100 and then rotated while advancing into the body 100 to expand the polygonal expansion part 120. The handle 200 is inserted into a rear end of the body 100, and may be screw-coupled to the passage part 110. Here, the rear end may be the other end in the longitudinal direction of the body 100 as the opposite side to the leading end described above.

Specifically, the handle 200 may include a rotating part 210, a stem part 220, and a pressing part 230.

The rotating part 210 may be configured to be gripped by hand. The rotating part 210 is provided so that a user can hold it by the hand and rotate the entire handle 200 with ease. As shown in FIGS. 5 and 6, the rotating part 210 may be configured in a disk shape or a bar shape as needed.

The stem part 220 may be connected to the rotating part 210 and rotated by receiving the rotational force of the rotating part 210. The stem part 220 may be inserted into the rear end of the body 100 to be screw-coupled to the passage part 110.

The stem part 220 may have a thread formed on an outer circumferential surface thereof to be screw-coupled with the thread of the passage part 110. Accordingly, when the rotating part 210 is rotated, the stem part 220 may move forward or backward along the passage part 110 in a state in which the stem part 220 and the passage part 110 are screw-coupled to each other.

In addition, the pressing part 230 is provided at the leading end of the stem part 220, and when the pressing part 230 reaches the tapered portion 121 of the polygonal expansion part 120 as the stem part 220 moves forward, the pressing part 230 may radially press the tapered portion 121. Accordingly, the polygonal expansion part 120 may be in close contact with the polygonal groove 21 of the locking screw 20 while expanding radially.

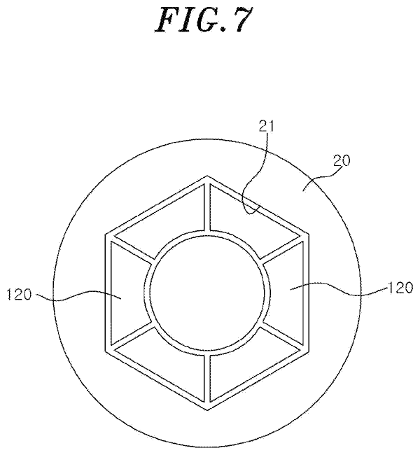

As shown in FIGS. 3 and 7, since no pressing force is applied to the tapered portion 121 before the pressing part 230 reaches the tapered portion 121 of the polygonal expansion part 120, the polygonal expansion part 120 is not expanded. Accordingly, a gap is formed between the polygonal expansion part 120 and the polygonal groove 21 of the locking screw 20, so that the polygonal expansion part 120 can be easily inserted into the polygonal groove 21.

That is, before expanded, the polygonal expansion part 120 can be easily inserted into the polygonal groove 21 of the locking screw 20, and after the polygonal expansion part 120 is inserted into the polygonal groove 21, a user rotates the rotating part 210 so that the stem part 220 moves forward toward the leading end of the body 100 along the passage part 110.

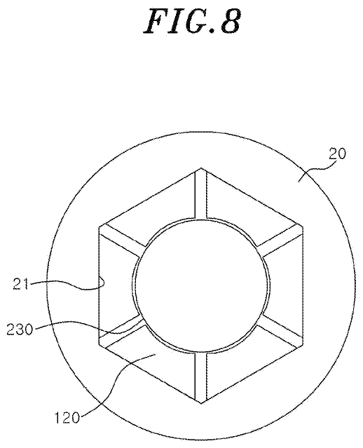

As the stem part 220 moves forward toward the leading end of the body 100, the pressing part 230 reaches the tapered portion 121 of the polygonal expansion part 120. In this state, when the rotating part 210 is further rotated, the polygonal expansion part 120 is expanded radially as the pressing part 230 presses the tapered portion 121, so that as shown in FIGS. 4 and 8, the polygonal expansion part 120 comes in close contact with the polygonal groove 21 of the locking screw 20.

In this state, when the rotating part 210 is further rotated or rotated together with the body 100, the rotational force is transferred to the locking screw 20 through the polygonal expansion part 120 to rotate the locking screw 20 in the releasing direction. As a result, the locking screw 20 can be easily removed from the bone.

It is common to use, as the locking screw 20, a right-hand screw that moves forward when rotated in the right direction (clockwise), and in this case, a rotational force in the left direction (counterclockwise) needs to be applied to the locking screw 20 in order to loosen the locking screw 20. Accordingly, the stem part 220 may be configured to be screw-coupled with the passage part 110 in a left-handed manner so as to move forward when the rotating part 210 is rotated counterclockwise.

Hereinafter, the operation of the present disclosure will be briefly described with reference to FIGS. 3 to 6.

In order to remove the locking screw 20 fixed to the bone in the orthopedic field, first, the stem part 220 of the handle 200 is inserted into the rear end of the body 100. When the stem part 220 is rotated counterclockwise in a state in which the stem part 220 is screw-coupled to the passage part 110 of the body 100, the pressing part 230 moves toward the leading end of the body 100 by screw coupling between the stem part 220 and the passage part 110.

When the pressing part 230 advances to some extent toward the leading end of the body 100, the polygonal expansion part 120 is inserted into the polygonal groove 21 of the locking screw 20 as in the state shown in FIGS. 3 and 5.

In this state, when the rotating part 210 is further rotated counterclockwise, the pressing part 230 radially presses the tapered portion 121 formed in the polygonal expansion part 120, and the pressing force is applied to the polygonal expansion part 120.

In addition, in this state, the polygonal expansion part 120 is also expanded radially as shown in FIGS. 4 and 6, and the polygonal expansion part 120 comes in close contact with the polygonal groove 21 of the locking screw 20 so that the body 100 would not be easily separated from the locking screw 20.

In addition, when the rotating part 210 and the body 100 are further rotated counterclockwise, the rotational force is transferred to the locking screw 20 through the polygonal expansion part 120 to rotate the locking screw 20 in the releasing direction. As a result, the locking screw 20 can be easily removed from the bone.

As described above, the present disclosure has been described using preferred embodiments. However, the scope of the present disclosure is not limited to the specific embodiments described, and those skilled in the art can easily change or modify the disclosed embodiments within a range of the present disclosure, which also belong to the scope of the present disclosure.

1-7. (canceled)

8. A surgical polygonal locking screw removal device, comprising:
a body including a passage part in which a passage having a preset diameter is formed, and a polygonal expansion part configured to be coupled to a polygonal groove formed in a surgical locking screw to remove the surgical locking screw, the polygonal expansion part protruding from a leading end of the passage part; and
a handle configured to be inserted into a rear end of the passage part and screw-coupled to the passage part and configured to expand the polygonal expansion part.

9. The surgical polygonal locking screw removal device of claim 8, wherein the polygonal expansion part is formed to be divided at regular intervals along a circumferential direction so that the polygonal expansion part is radially expandable when inserted into the polygonal groove of the surgical locking screw.

10. The surgical polygonal locking screw removal device of claim 9, wherein cutouts are formed in the leading end of the passage part.

11. The surgical polygonal locking screw removal device of claim 8, wherein the polygonal expansion part includes a tapered portion which communicates with the passage part and an inner diameter of which is gradually decreased toward a leading end of the polygonal expansion part.

12. The surgical polygonal locking screw removal device of claim 11, wherein the handle includes:
a rotating part configured to be gripped by a hand;
a stem part connected to the rotating part, the stem part configured to be inserted into the rear end of the passage part and screw-coupled to the passage part to move forward or backward along the passage part when the rotating part is rotated; and
a pressing part provided at a leading end of the stem part and configured such that when the pressing part reaches the tapered portion of the polygonal expansion part as the stem part moves forward, the pressing part radially presses the tapered portion to expand the polygonal expansion part to be close contact with the polygonal groove.

13. The surgical polygonal locking screw removal device of claim 12, wherein the stem part is screw-coupled to the passage part in a left-handed manner to move forward when the rotating part is rotated counterclockwise.

14. The surgical polygonal locking screw removal device of claim 10, wherein each of the cutouts and gaps formed between the divided portions of the polygonal expansion part is arranged on a line.
